(19) United States
(12) Patent Application Publication
(10) Pub. No.: US 2025/0120586 A1
(43) Pub. Date: Apr. 17, 2025

(54) ORAL HEALTH SELF-MONITORING SYSTEM

(71) Applicant: Smylio Inc., Fremont, CA (US)

(72) Inventors: Varghese George, Trivandrum (IN); Renjith Menon, Campbell, CA (US)

(21) Appl. No.: 18/913,771

(22) Filed: Oct. 11, 2024

Related U.S. Application Data
(60) Provisional application No. 63/590,571, filed on Oct. 16, 2023.

(51) Int. Cl. A61B 1/24; A61B 1/00; A61B 1/04; A61B 1/06; A61B 1/32

(52) U.S. Cl. CPC A61B 1/24; A61B 1/000096; A61B 1/00016; A61B 1/00105; A61B 1/00147; A61B 1/04; A61B 1/06; A61B 1/32

(57) ABSTRACT

An oral health self-monitoring system includes a portable photo guide that is adapted to cooperate with a smartphone camera to obtain images of the patient's oral cavity. A processor, preferably programmed and operable to execute machine learning algorithms, analyzes the images for cavities and plaque. In some implementations, the portable photo guide includes an onboard camera, and the smart phone is connected to the camera and programmed to manage the camera to obtain images of the patient's oral cavity and to send them to a remote server for analysis.

### CROSS-REFERENCE TO RELATED APPLICATIONS

This claims priority to provisional patent application No. 63/590,571, filed Oct. 16, 2023, and entitled “ORAL HEALTH SELF-MONITORING SYSTEM”, the entirety of which is incorporated by reference in its entirety for all purposes.

### FIELD OF THE INVENTION

The subject matter of the present disclosure relates generally to the field of oral health. More particularly, the present disclosure relates to an apparatus and method for detecting cavities, plaque or tartar.

### BACKGROUND

Currently, it is challenging for patients to monitor their oral wellness at home for a number of reasons: it is difficult to visualize inside one's own oral cavity; cavities are not visible until too late, and often, only after pain is present; and plaque is difficult to see and evaluate by eye.

A novel apparatus and method that overcomes the above-mentioned challenges is therefore desirable.

### SUMMARY OF THE INVENTION

Embodiments of the invention include a system for monitoring oral health at home, and preferably for detecting caries or plaque.

In embodiments of the invention, a portable guide or apparatus is operable with a smartphone to take photos of the user's teeth under various conditions.

In embodiments of the invention, a camera guide has a front window that is aligned with the smartphone camera for aiming the camera into the oral cavity. Optionally, the guide comprises a ridge or shelf for the smart phone to be mounted.

In embodiments of the invention, the guide comprises a strap to secure the smartphone thereto.

In embodiments of the invention, the guide includes lights directed at the oral cavity, and optionally, lights operable at a wavelength range that induces fluorescence, increases contrast, highlights or otherwise more visibly shows plaque or caries versus the natural tooth.

In embodiments of the invention, the processor is programmed and operable to evaluate brightness, color, or contrast of an area of an image for a threshold value indicative of a cavity, tartar, or an implant or crown.

In embodiments of the invention, a processor is programmed, preferably, using artificial intelligence (AI) algorithms to detect caries or plaque based on the images.

In embodiments of the invention, a camera and optionally processor are built into the guide.

In embodiments, an oral health self-monitoring system for use with a camera comprises a camera guide and a camera adapter to support the camera and to aim the camera into the oral cavity.

In embodiments, the camera adapter is shaped to house a camera.

In embodiments, the camera adapter is detachable from the guide.

In embodiments, the system further comprises a strap to couple the smart phone to the camera adapter.

In embodiments, the system further comprises at least one light source for emitting light, and wherein the camera is operable to obtain images through the camera guide.

In embodiments, the system further comprises a processor programmed and operable to determine oral health of the user based on the images, wherein the oral health includes evaluating for the presence of at least one selected from the group consisting of caries, plaque, tartar, and implants.

In embodiments the processor is remote or cloud-based.

In embodiments, the system further comprises a portable computing device programmed and operable to manage the camera for taking and obtaining the images, and for sending the images to the processor for determining the oral health of the user. In embodiments, the portable computing device is a smart phone and runs an App for managing the camera.

In other embodiments, the camera adapter has a hollow tubular shape and a front window, and the camera adapter is arranged with the smart phone to align the smart phone camera with the front window in the camera adapter when the smart phone is secured to the camera adapter.

In embodiments, a kit comprises camera guide and two different camera adapters including a first camera adapter comprising a housing for enclosing the camera and a second camera adapter comprising a hollow tubular body for coupling to a smart phone camera.

In embodiments of the invention, a non-transitory storage medium for monitoring oral heath, and having a set of computer-readable instructions stored thereon, is operable with a processor to receive images of the oral cavity and to analyze the images for oral health including analyzing the images for caries, tartar, and plaque.

An advantage of embodiments of the invention arises from fixedly arranging the camera (whether a dedicated camera or smart phone camera) with the guide such that the user can take photos of the teeth having consistent high quality. In embodiments, the user can advance the mouth retractor into the mouth of the user and the camera is automatically in position to take a photo without the user being required to independently manipulate the camera relative to the guide or teeth. In a sense, the camera-mounted guide provides single-handed operation.

### BRIEF DESCRIPTION OF THE DRAWINGS

For a better understanding of at least certain embodiments, reference will be made to the following Detailed Description, which is to be read in conjunction with the accompanying drawings where:

FIG. 1 is a flow diagram of a process, according to some embodiments of the invention;

FIG. 2 is a perspective view of a guide, according to some embodiments of the invention;

FIG. 3 is a schematic illustration of an oral health monitoring system, according to embodiments of the invention.

FIG. 4 is a perspective view of a guide including a smart phone strap, according to some embodiments of the invention;

FIG. 5 is a perspective view of an oral health self-monitoring system including a guide and a camera assembly, according to some embodiments of the invention;

FIG. 6 is an exploded view of the oral health self-monitoring system shown in FIG. 5;

FIG. 7 is a top view of the camera housing shown in FIG. 5 without the camera;

FIG. 8 is top view of the camera housing shown in FIG. 5 including the camera;

FIG. 9 is a top view of a phone adapter; and

FIGS. 10A-10C are, respectively, raw or normal light, UV light-enhanced, and preprocessed images of a user's oral cavity obtained using a camera guide assembly in accordance with embodiments of the present invention.

The figures depict various embodiments of the present invention for purposes of illustration only, wherein the figures use like reference numerals to identify like elements. One skilled in the art will readily recognize from the following discussion that alternative embodiments of the structures and methods illustrated in the figures may be employed without departing from the principles of the invention described herein.

### DETAILED DESCRIPTION

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

All existing subject matter mentioned herein (e.g., publications, patents, patent applications and hardware) is incorporated by reference herein in its entirety except insofar as the subject matter may conflict with that of the present invention (in which case what is present herein shall prevail).

### Overview of Process

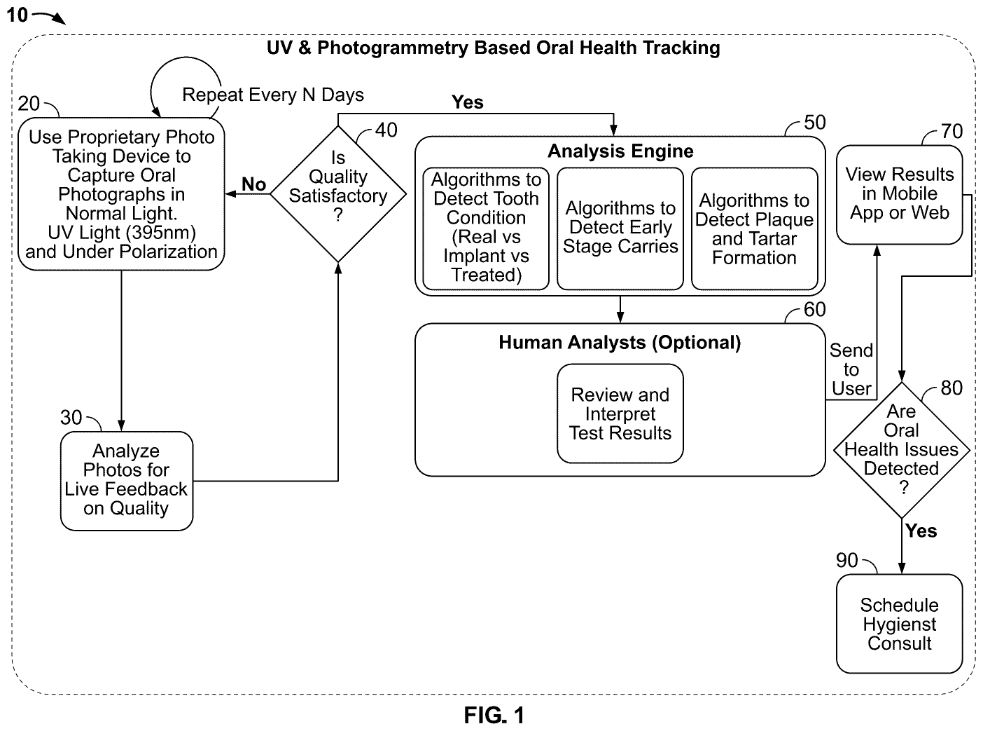

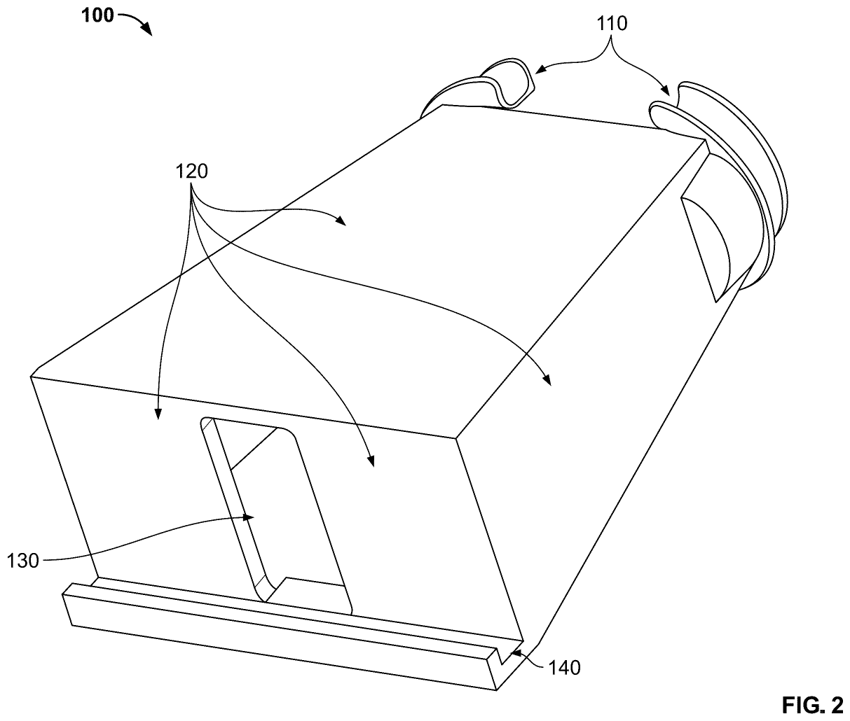

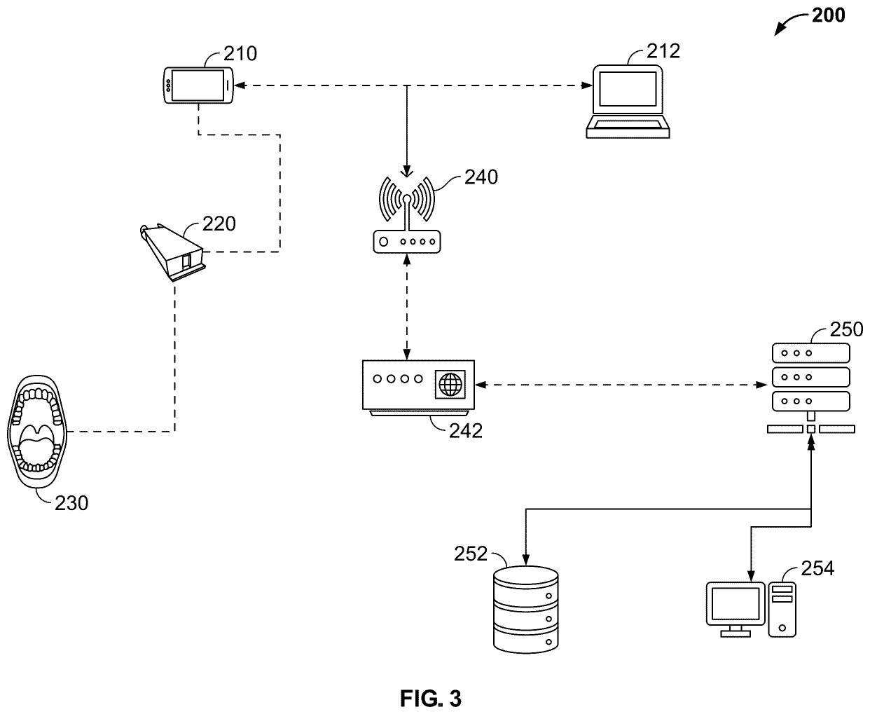

FIG. 1 is a flowchart of a process 10 in accordance with embodiments of the invention. As the process 10 is described reference shall also be made to FIGS. 2 and 3 for understanding of exemplary components for carrying out some of the steps described in process 10. The process 10 shown in FIG. 1 can be used for continuous wellness monitoring & orthodontic movement tracking of a patient using AI, heuristic algorithms, and human intelligence to proactively catch health issues and give the patient actionable insights.

Step 20 states to use proprietary device to capture photographs of the oral cavity in normal light, and UV light (e.g., about 395 nm), and preferably under polarization. In embodiments, a guide 100 as shown in FIG. 2 is provided to support and aim a camera (optionally of a smart phone) at the oral cavity of the patient. A downloaded App on the smart phone is operable to work with the phone's camera to obtain photos of the oral cavity.

In embodiments, lights associated with the smart phone are directed at the target. In other embodiments, lights associated with the guide are directed at the target. For embodiments, the guide 100 includes lights directed at the oral cavity, and optionally, lights operable at a wavelength range that induces fluorescence, increases contrast, highlights or otherwise more visibly shows plaque or caries versus the natural tooth. In preferred embodiments, the lights of the guide are controlled by the App of the smart phone using wireless technology such as, e.g., Bluetooth.

As described further herein with reference to FIG. 2, the guide 100 comprises a mouth retractor 110 to hold open the patient's mouth and provides visibility to the oral cavity for the camera.

Step 30 states to analyze photos for live or real-time feedback on quality. In embodiments, the smart phone is programmed and operable to perform this step. Each photo may be evaluated for minimum resolution and image attributes.

Step 40 queries whether the quality of the photos is satisfactory. For example, each photo is evaluated for whether the image attributes are below a threshold value. If the image quality attribute (e.g., resolution, contrast, etc.) is below a threshold, the user is instructed to repeat or retake the photos.

If the photos meet minimum quality, the process proceeds to step 50 for the images to be analyzed in the analysis engine.

The minimum quality may be determined based on various properties or parameters. For embodiments, for example, the minimum quality is based on blur detection and an algorithm such as the variance of Laplacian of Gaussian (LoG), where a value above a threshold of about 3 is deemed acceptable. For embodiments, the minimum quality is based on resolution, and an acceptable resolution is a value greater than 3 megapixels (MP). It is also to be understood that the threshold values can change depending on the exact sensor and lighting used. Calibration of the system is preferably performed when a new camera or lighting is implemented.

In embodiments, if the images are acceptable, the images are preprocessed. For example, preprocessing could include applying bandpass filters or otherwise adjusting the light, hue, saturation, and other properties of the image. In embodiments, preprocessing includes one or more of the following: reducing the lightness values (e.g., <−10 units) and saturation values (e.g., <−25 units) in the blue wavelength range, boosting the lightness values (e.g., >25 units) and saturation values (e.g., >35 units) in the red wavelength range, and (c) reducing the hue values (e.g., <−40 units) and saturation values (e.g., <−20 units) in the cyan wavelength range.

In the embodiment shown in FIG. 1, an analysis engine 50 comprises a plurality of different algorithms or models to evaluate the photos including (a) an algorithm to detect tooth condition (e.g., implant, real, or treated), (b) an algorithm to detect early-stage caries, and (c) an algorithm to detect plaque and tartar formation.

In embodiments, the analysis is performed on one or more servers (e.g., a cloud-based server) by the models.

In embodiments, an analysis engine includes a segmentation phase and a detection phase. For embodiments, the system includes an object segmentation model such as, for example, YOLO, in combination with heuristic rules to combine overlapping areas and assign tooth names to the segmented areas.

Next, the data is sent to the respective object detection models for carries detection and tartar detection. Each of the tooth areas detected is also sent to a classifier model to determine if the tooth is an implant/crown or natural tooth. This classifier may work on the tooth areas either in isolation or together to be able to compare relative features such as brightness.

In embodiments, one or more of the models is a trained machine learning model, (e.g., a convolution neural network model such as YOLO) operable to provide a score, probability, or otherwise classify tooth condition (real, implant, and treated), caries, and plaque for each tooth based on the images provided by the smart phone or camera as the case may be.

In embodiments, each model is rule based and not a machine learning algorithm. For example, each model may be operable to interrogate the segmented image area for ‘hot spots.’ In embodiments, the model applies image thresholding for determining the presence of caries, tartar, plaque, or a tooth implant or crown. If the value for a pixel area exceeds a threshold value, the area is deemed a ‘hot spot’ corresponding to caries, tartar or crown or tooth replacement. As described herein, the color or brightness can be indicative of the type of defect.

Step 60 states to review and interpret the results. This step can be optional, and performed by a human analyst. The human analyst may review the photographs, and the scores from the analysis engine, and confirm, edit, or otherwise modify the results.

Step 70 states to view the results. This step may be performed by the smart phone, computer, or another device operable to receive the results from step 50 or step 60. Examples of other devices to display the results from the analysis engine are internet-connected (whether by Wi-Fi or otherwise) tablets, smart phones, laptops, desktops, etc.

Step 80 queries whether oral health issues are detected. This step can be performed by the user or doctor or one of the programmed devices based on the test results. In embodiments, this step is automatic and the smart phone or user device is programmed to provide an alert to the user/patient of an oral health issue. Examples of alerts include SMS, audio sounds, email, and badges.

Step 90 states to schedule an appointment. This step may be performed by the user. In other embodiments, an alert is sent to the hygienist for the hygienist to contact the patient for scheduling an appointment.

### Portable Guide

With reference to FIG. 2, a portable apparatus or guide 100 is shown in accordance with the present invention. It includes an integrated cheek retractor 110 to maintain the mouth wide open, and provide a clear shot of the oral cavity; and a body portion comprising opaque walls 120 to prevent light leaks. The body portion is shown having a generally rectangular cross section. The length of the body portion may vary. An exemplary length of the body portion is greater than 80 mm, and more preferably from 80-160 mm, more preferably 80-120 mm.

The front walls could optionally incorporate LEDs directing light rearward towards the mouth piece and teeth to enable photographing under different light conditions. The side or rear walls could optionally include calibration markers to calibrate for scale, skew and color.

In embodiments of the invention, the cheek retractor 110 is detachable from the body portion. A cheek retractor may be provided to accommodate a particular mouth size (e.g., small or child, medium, large, etc.). In embodiments of the invention, a kit includes a plurality of different size cheek retractors to accommodate various mouth sizes.

The apparatus is also shown including a viewport 130 adapted to cooperate with the smartphone or mobile camera and flash to be in line with the oral cavity.

A channel 140 is adapted to hold the phone in a stable position.

An advantage of embodiments of the invention is image quality. The image quality is enhanced by use of the guide described herein and arranging the camera a fixed distance from the mouth. Focusing, distancing, and darkness are predictable and ‘built in’ to the system to operate effectively. Namely, the shape, length and arrangement of the retractor, guide body and shelf or front end operate to optimally locate the camera from the mouth under optimal conditions for obtaining high quality images of the teeth. Embodiments of the invention build in repeatability without being dependent on the skill of the user in controlling hand motion and image quality. This is an advantage because different users can have different skill levels and ability levels for positioning the camera and taking photos.

In embodiments of the invention, the system takes photos of the patient's teeth under normal light and under UV light. As described above, the images are then analyzed by a processor (local or remote) programmed and operable to identify areas of interest such as dental tartar formation or caries. In embodiments, the processor is programmed with AI algorithms and/or human intelligence and heuristic algorithms to identify the areas of interest. In embodiments, image thresholding is performed for detection of defects. For embodiments, RGB or HUE thresholding can be applied for computing hot spots in the images.

Without intending to be bound to theory, dental tartar should fluoresce orange under long wave UV light (around 395 nm range). The specific polymorphs of calcium carbonate that make up dental tartar is the reason for this fluorescence.

Similarly, certain dental conditions like caries (namely, cavities) will have increased contrast under UV light.

The UV light can also be used to identify which of the teeth has had dental corrective work such as crowns (or implants) because the tooth with natural enamel (lacking corrective work) will glow a brighter (almost yellowish) color versus the tooth with corrective work such as crowns (or implants) will appear darker. Knowing the presence and location of the corrective work is relevant for planning certain dental procedures such as orthodontic treatments.

### Hardware Overview

FIG. 3 is a schematic illustration of an oral health monitoring system 200, according to embodiments of the invention. The system 200 is shown including smart phone 210 that operably cooperates with guide 220 as described above for obtaining images of the oral cavity 230 of the patient.

The system 200 further includes a Wi-Fi-enabled router 240 connected to internet via modem 242.

A cloud-based server 250 is operable to send and receive data and software updates via the modem and router to the smart phone 210, and optionally a user computer or laptop 212.

All data and algorithms and results may be stored in database 252.

A backend-type computer 254 is shown in the system 200 to allow a hygienist or analyst to access the data, results, and algorithms. Access is useful to carry out various actions including without limitation: reviewing of the results, annotating the data, and updating or providing maintenance to the App and analysis engines.

In embodiments, the server 250 is programmed and operable to host a website, allowing the patient or another type of user to register, login, and access the data and results stored in the database via an internet-connected computer, workstation, desktop, smartphone 210, or laptop computer 212. The user can input a wide range of information including, for example, personal identification information, health information and history, current care and preventive maintenance and habits, etc. for use to evaluate the health of the oral cavity. Such features can be inputs to the machine learning algorithms for detecting tooth health, caries, corrective work, and plaque.

### Alternative Embodiments

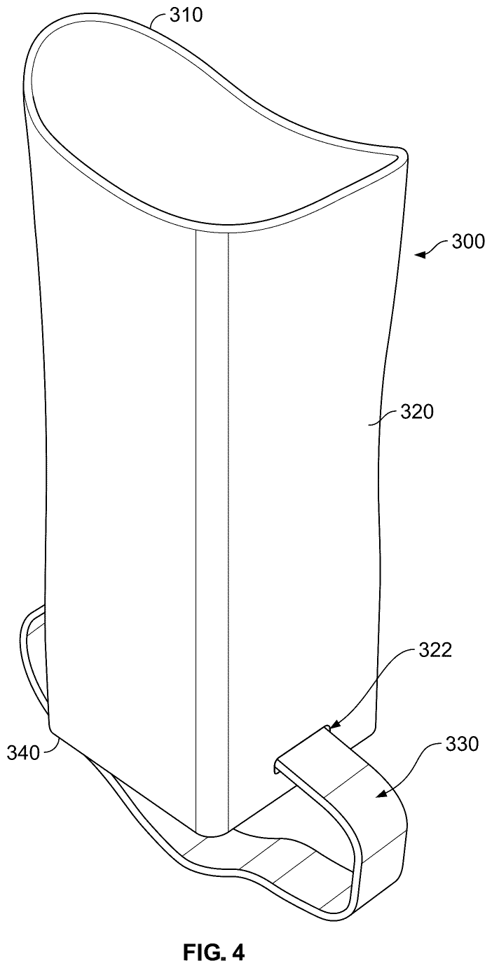

FIG. 4 shows an alternative embodiment of a portable guide 300 in accordance with embodiments of the invention. The guide 300 is similar to the guide 200 shown in FIG. 2 except guide 300 includes a strap 330 for coupling the smart phone or camera to the guide body 320. The front 340 can include an opening or view port for the camera. Lights may be incorporated into the front wall for illuminating the teeth. Optionally, a shelf or channel (not shown) may be incorporated into the front 340 for further positioning the camera.

Fixedly locating the camera lens a predetermined distance from the user's teeth serves to control and optimize distance and focus. Additionally, mounting the phone or camera to the front view port serves to control lighting/darkness since external light is prohibited from entering the guide channel.

In the embodiment shown in FIG. 4, the cheek retractor 310 has a low-profile and is incorporated into the body of the guide. However, it is to be understood that the guide may vary widely and features may be added, omitted, and combined in any logical combination where such features are not exclusive to one another.

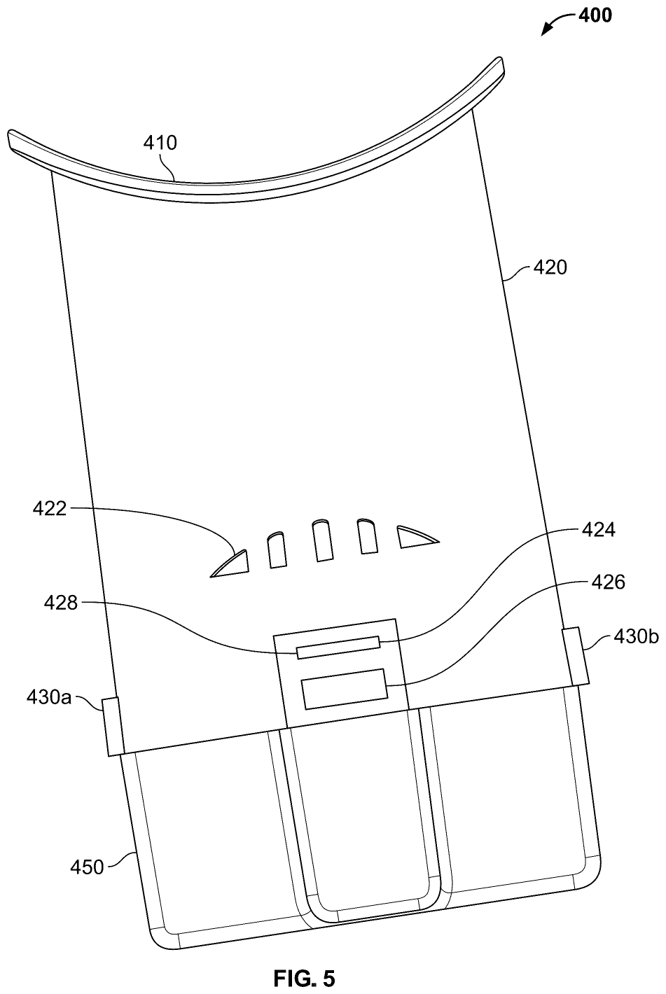

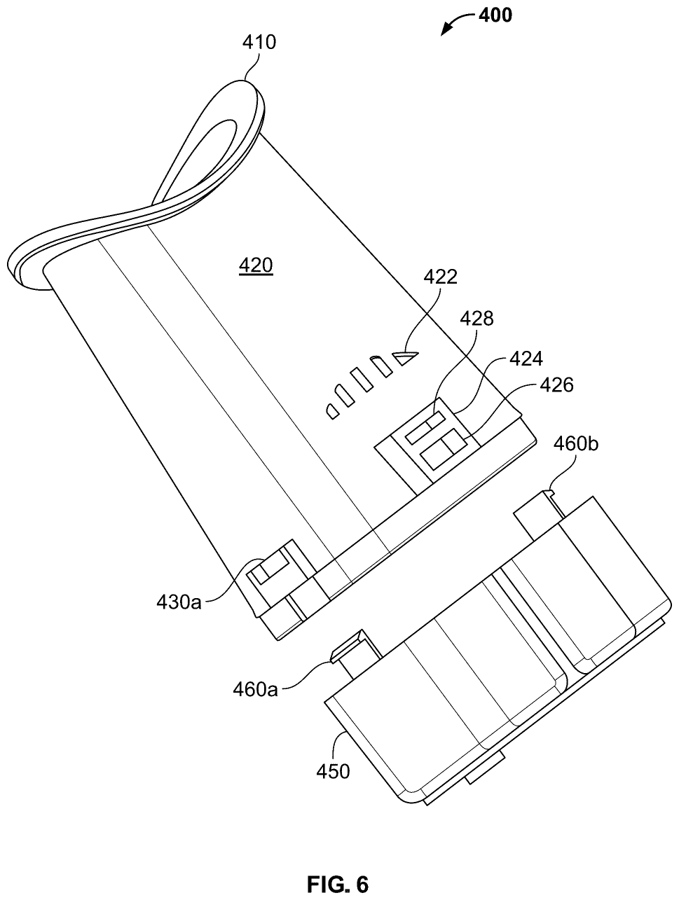

FIGS. 5-6 show an oral health monitoring system 400 in accordance with another embodiment of the invention with an integrated camera assembly.

In embodiments, the system includes an integrated cheek retractor-style mouthpiece 410, a tubular body 420, vent holes 422 serving to prevent fog build up from the user's breath, and camera housing 450.

In embodiments, the camera housing 450 contains a rechargeable camera, optionally, with lights (e.g. LEDs) for illuminating the target features at selected wavelengths as described above.

In embodiments, the camera is connected to another computing device (e.g., a smartphone, tablet or computer) either via cable (e.g., USB) or wirelessly (e.g., via WIFI or Bluetooth). The computing device is programmed and operable to, as described above, execute a program to carry out the steps set forth in FIG. 1, except the dedicated camera generates the images instead of the smart phone.

In embodiments, the local computing device (e.g., the smart phone or tablet) receives the image data and sends the data to another device (e.g., remote server) for performing the analysis. Then, the backend server performs the analysis and returns the results to the user on a website or mobile App.

Optionally, the camera housing 450 includes cable cutouts for wired connections or includes ports for charging onboard rechargeable batteries. For example, a power socket such as a USB-C port can be implemented in the camera housing to charge the camera and electronics.

An elastic band (not shown) may be connected to the system via two openings 424, 426. The openings are connected to one another by a channel. The band may be connected through the openings and channel such that its length is adjustable.

With reference to FIG. 6, the camera housing 450 is shown separated from the guide tube 420. The camera housing is releasably connectable to the guide tube 420 via tabs 460a, 460b and recesses' 430a, 430b. In the embodiment shown in FIG. 6, the tabs are deflectable and snap into the recesses' when the camera housing 450 is fully engaged with the guide tube 420. The removable base allows the channel tube 420 (and mouth piece 410 if integrated with the tube) to be cleaned separately and easily from the camera assembly 450 without damaging the electronics.

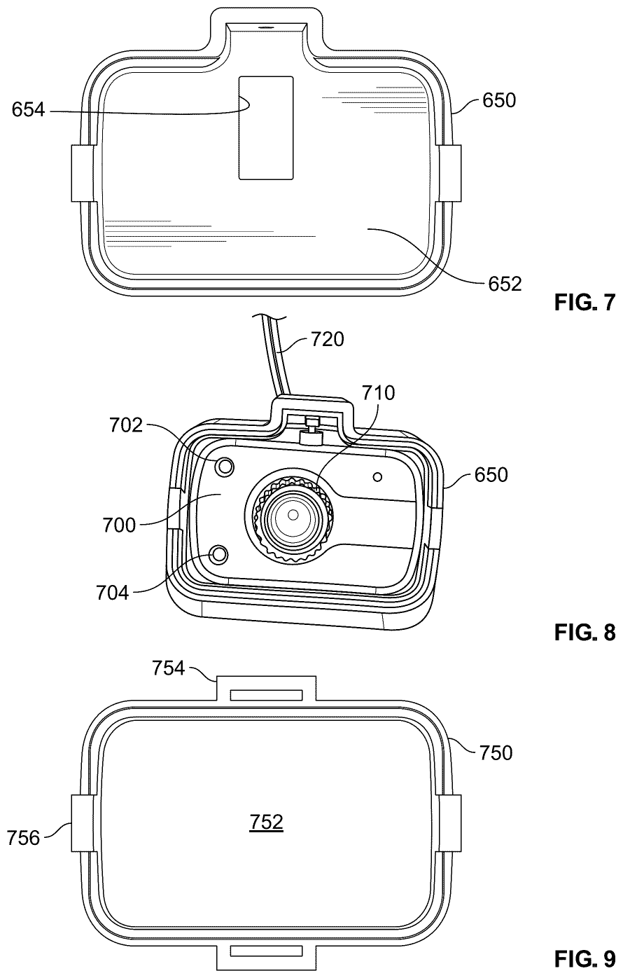

FIG. 7 is a top view of a camera assembly housing in accordance with embodiments of the invention. It includes a body or walls 650, floor 652, and a cutout 654. The cutout can be used for accommodating the cabling between the camera and the computing devices or power supply, as described herein.

FIG. 8 is a top view of a camera assembly housing 650 and camera 700 disposed therein in accordance with embodiments of the invention. The camera is shown including LED emitters 702, 704, lens 710, and cable 720 extending from below. The cable may be arranged to connect to the camera through a cutout 654 as described above in connection with FIG. 7.

An example of a camera is the Zeb-Crystal Clear Camera with 3P lens, night vision emitters, and a CMOS image sensor by Zebronics India Pvt. Ltd. (Vepery, Chennai, Tamil Nadu, India).

FIG. 9 is a top view of a phone adapter in accordance with embodiments of the invention. It includes a body or walls 750 defining a rectangular open passageway 752 for the camera of a smart phone to be aligned. Tabs 756 are shown on the exterior of the left and right walls for engaging the channel guide as described above. Band connectors 754 are shown on the exterior of the upper and lower walls. A band can be looped around a phone and fastened to the connectors 754 for securing a smart phone to the adapter. The smart phone can be used to obtain images of the oral cavity, interrogate the images for defects, and to monitor oral health of the user.

Light source assemblies could be incorporated into the walls of the adapter and aimed rearward towards the mouth piece. For example, small low profile LED assemblies could be incorporated into the walls or body of the adapter so as to avoid interfering or obstructing the camera view and illuminate the teeth. Preferably, as described herein, UV light is aimed at the teeth.

It should be apparent that the phone adapter 750 can be swapped for the camera assembly housing 650 (and vice versa). In either configuration, images of the oral cavity can be collected and evaluated to monitor oral health of the user.

In embodiments, after the smart phone or camera assembly is located or mounted to the guide, a calibration is performed to calibrate the camera and lighting. In embodiments, calibration markers are arranged on the inner side wall towards the rear of the guide body. The markers may be used for correcting lens distortion, measuring size of the objects, segmentation, and color calibration. Calibrating the camera improves accuracy.

In alternative embodiments, an algorithm or model is applied to detect conditions visible under RGB light including gingivitis, gum recession, visible carries, etc.

In embodiments, a method for monitoring oral health of the user comprises any one or more of the steps described herein to detect caries, plaque, tartar and implants such as, for example, crowns.

### Example

A camera guide as described above in connection with FIG. 2 was arranged with a Google Pixel 6 smart phone, aligning its (12.5 MP) rear camera with the view port in the camera guide for taking an image of a user's mouth. LEDs were incorporated into the front wall of the camera guide to the left and right of the view port so as to not interfere with the view of the camera.

The mouth piece was arranged within the mouth of the user, teeth closed.

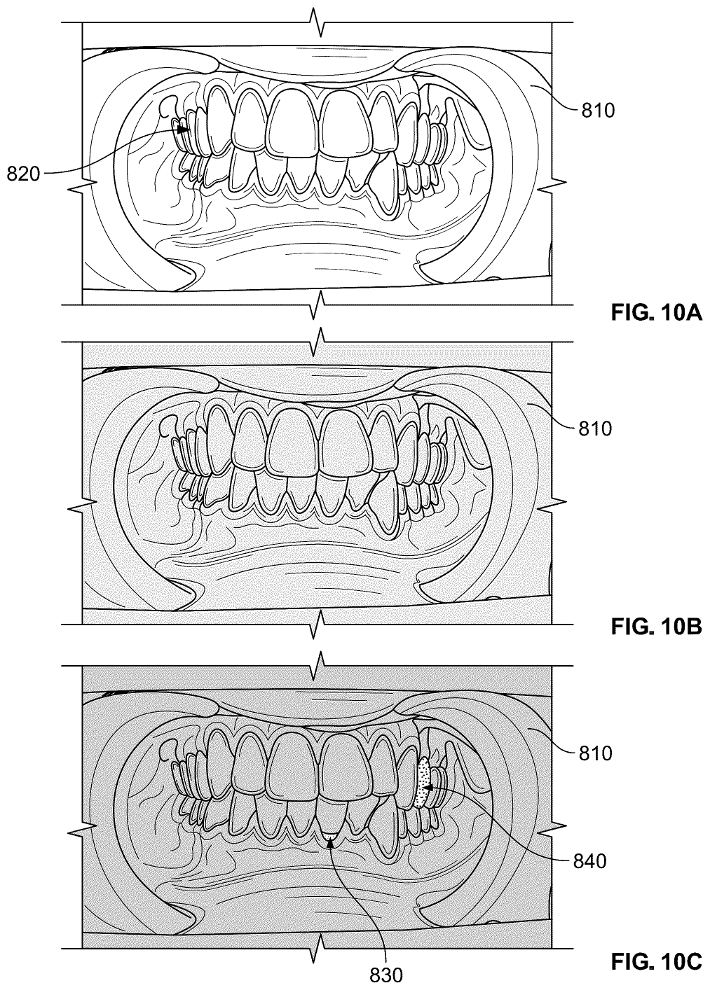

FIG. 10A shows the raw image taken under normal light. That is, the camera assembly emitted light through the guide in the normal visible light range into the mouth. Although gaps between the teeth and gum line are visible, defects or tooth health is hard to detect.

FIG. 10B shows same shot of the user's teeth under night vision. In this example, the camera assembly emitted light in the UV range of 390-400 nm into the mouth. In this image, we can see that certain spots appear different than others. For example, the rear right side upper tooth is darker than the others. However, this effect can be enhanced by application of filters and preprocessing, discussed herein.

FIG. 10C shows the UV image from FIG. 10B after preprocessing has been applied. In this example, preprocessing included (a) reducing the lightness values (e.g., −13 units) and saturation values (e.g., −27 units) in the blue wavelength range, (b) boosting the lightness values (e.g., 27 units) and saturation values (e.g., 35 units) in the red wavelength range, and (c) reducing the hue values (e.g., −43 units) and saturation values (e.g.,-24 units) in the cyan wavelength range.

Various features are clearly visible. For example, tartar 830 glows reddish orange under UV. A fake tooth 840 doesn't fluoresce the same way as real enamel and shows up as a dull color.

Other things we could see include minor carries. Minor carries show as dark spots—the undamaged tooth enamel around the spot is brighter under UV.

The UV image also makes segmentation of teeth easier giving a higher contrast image.

The above image data are evidence that embodiments of the subject invention can monitor oral health of a user. The device has the advantage of having self-serving functionality for use at home with the user's smart phone or tablet or computer.

Throughout the foregoing description, and for the purposes of explanation, numerous specific details are set forth in order to provide a thorough understanding of the described techniques. It will be apparent, however, to one skilled in the art that these techniques can be practiced without some of these specific details. Although various embodiments that incorporate these teachings have been shown and described in detail, those skilled in the art could readily devise many other varied embodiments or mechanisms to incorporate these techniques.

For example, although the analysis engine was described being on a server, in some embodiments of the invention, the analysis engine is a submodule of the App on the smartphone. In embodiments, an App on the smartphone is operable to perform all the steps including taking the images, processing and analyzing the images, saving and displaying the results.

Also, embodiments can include various operations as set forth above, fewer operations, or more operations; or operations in an order. Accordingly, the scope and spirit of the invention should be judged in terms of the claims, which follow as well as the legal equivalents thereof.

1. An oral health self-monitoring system for use with a smartphone camera comprises a camera guide to support the camera and to aim the camera into the oral cavity.

2. The system of claim 1, further comprising a processor programmed and operable to determine the oral health based on images received from the camera of the oral cavity.

3. The system of claim 2, further comprising a UV light source, and wherein the system is operable to aim UV light into the oral cavity.

4. The system of claim 3, wherein the processor is programmed and operable to compute the oral heath using a trained machine learning model.

5. The system of claim 3, wherein the camera guide comprises the UV light source to emit the UV light.

6. The system of claim 4, wherein the processor is a component of a server in communication with the smart phone.

7. The system of claim 1, wherein the camera guide comprises a mouth retractor.

8. The system of claim 4, wherein the images comprise RGB images, and the system comprises a first model for detecting locations of each of the teeth using RGB images.

9. The system of claim 8, comprising a second model for evaluating the health of each of the teeth locations based (a) on the locations detected using the first model and (b) the UV images.

10. The system of claim 1, further comprising a member to hold the smart phone to the camera guide, and wherein the member is a shelf, channel or band.

11. An oral health self-monitoring system for use with a camera comprises a camera guide and a camera adapter to support the camera and to aim the camera into the oral cavity.

12. The system of claim 11, further comprising the camera, and wherein the camera adapter is shaped to house a dedicated camera.

13. The system of claim 11, wherein the camera is a smart phone camera, and wherein the camera adapter has a hollow tubular shape and a front window, and the camera adapter mechanically cooperates with the smart phone to align the smart phone camera with the front window in the camera adapter when the smart phone is secured to the camera adapter.

14. The system of claim 11, wherein camera adapter is detachable from the guide.

15. The system of claim 13, further comprising a strap to couple the smart phone to the camera adapter.

16. The system of claim 12, further comprising at least one light source for emitting light, and wherein the camera is operable to obtain images through the camera guide.

17. The system of claim 16, further comprising a processor programmed and operable to determine oral health of the user based on the images, wherein the oral health includes evaluating for the presence of at least one selected from the group consisting of caries, plaque, tartar, and implants.

18. The system of claim 17, wherein the processor is remote or cloud-based.

19. The system of claim 18, further comprising a portable computing device programmed and operable to manage the camera for taking and obtaining the images, and for sending the images to the processor for determining the oral health of the user.

20. The system of claim 19, wherein the portable computing device is a smart phone, tablet, or laptop.
